(19) United States
(12) Patent Application Publication
(10) Pub. No.: US 2021/0393166 A1
(43) Pub. Date: Dec. 23, 2021

(54) Monitoring user health using gait analysis

(71) Applicant: Apple Inc., Cupertino, CA (US)

(72) Inventors: Matthew S. DeMers, Mountain View, CA (US); Edith M. Arnold, San Francisco, CA (US); Adeeti V. Ullal, Mountain View, CA (US); Vinay R. Majjigi, Mountain View, CA (US); Mariah W. Whitmore, Cupertino, CA (US); Mark P. Sena, Larkspur, CA (US); Irida Mance, Palo Alto, CA (US); Richard A. Fineman, Campbell, CA (US); Jaehyun Bae, San Carlos, CA (US); Maxsim L. Gibiansky, Sunnyvale, CA (US); Gabriel A. Blanco, San Francisco, CA (US); Daniel Trietsch, Cupertino, CA (US); Rebecca L. Clarkson, San Francisco, CA (US); Karthik Jayaraman Raghuram, Mountain View, CA (US)

(21) Appl. No.: 17/356,355

(22) Filed: Jun. 23, 2021

Related U.S. Application Data
(60) Provisional application No. 63/042,779, filed on Jun. 23, 2020.

(51) Int. Cl. A61B 5/11; A61B 5/00

(52) U.S. Cl. CPC A61B 5/112; A61B 5/1121; A61B 2560/0257; A61B 5/7278; A61B 2562/0219; A61B 5/1126

(57) ABSTRACT

In an example method, a computing device obtains sensor data generated by one or more accelerometers and one or more gyroscopes over a time period, including an acceleration signal indicative of an acceleration measured by the one or more accelerometers over a time period, and an orientation signal indicative of an orientation measured by the one or more gyroscopes over the time period. The one or more accelerometers and the one or more gyroscopes are physically coupled to a user walking along a surface. The computing device identifies one or more portions of the sensor data based on one or more criteria, and determines characteristics regarding a gait of the user based on the one or more portions of the sensor data, including a walking speed of the user and an asymmetry of the gait of the user.

### CROSS-REFERENCE TO RELATED APPLICATION

This application claims priority to U.S. Provisional Patent Application No. 63/042,779, filed Jun. 23, 2020, the entire contents of which are incorporated herein by reference.

TECHNICAL FIELD

The disclosure relates to techniques for electronically monitoring a user's health by analyzing the user's gait.

### BACKGROUND

An accelerometer is a device that measures the acceleration experienced by an object (e.g., the rate of change of the velocity of the object with respect to time). A gyroscope is a device that measures the orientation of an object. In some cases, a mobile electronic device (e.g., a cellular phone, a smart phone, a tablet computer, a wearable electronic device such as a smart watch, etc.) can include one or more accelerometers that determine the acceleration experienced by the mobile electronic device over a period of time and/or one or more gyroscopes that measure the orientation of the mobile electronic device. If the mobile electronic device is secured to a user, the measurements obtained by the accelerometer and the gyroscope can be used to approximate the acceleration experienced by the user over the period of time and the orientation of a body part of a user, respectively.

### SUMMARY

Systems, methods, devices and non-transitory, computer-readable mediums are disclosed for electronically monitoring a user's health by analyzing the user's gait.

In an aspect, a method includes obtaining, at a computing device, sensor data generated by one or more accelerometers and one or more gyroscopes over a time period. The sensor data includes an acceleration signal indicative of an acceleration measured by the one or more accelerometers over a time period, and an orientation signal indicative of an orientation measured by the one or more gyroscopes over the time period. The one or more accelerometers and the one or more gyroscopes are physically coupled to a user walking along a surface. The method also includes identifying, by the computing device, one or more portions of the sensor data based on one or more criteria; and determining, by the computing device, characteristics regarding a gait of the user based on the one or more portions of the sensor data, where the characteristics include a walking speed of the user and an asymmetry of the gait of the user.

Implementations of this aspect can include one or more of the following features.

In some implementations, the characteristics can include a step length of the user.

In some implementations, the characteristics can include a percentage of time that both feet of the user are contacting the ground during a cycle of the gait of the user.

In some implementations, the method can also include determining, based on the sensor data, the acceleration with respect to an inertial frame of reference.

In some implementations, the characteristics regarding a gait of the user can be estimated based on a pendulum model having the acceleration signal as an input.

In some implementations, the one or more portions of the sensor data can be identified based on an estimated grade of the surface.

In some implementations, the grade of the surface can be estimated based on a barometer measurement obtained from a barometric sensor.

In some implementations, the one or more portions of the sensor data can be identified based on a comparison between the acceleration signal and a simulated acceleration signal.

In some implementations, the simulated acceleration signal can be determined based on a pendulum model.

In some implementations, the one or more portions of the sensor data can be identified based on an estimated activity type of the user during the time period.

In some implementations, one or more portions of the sensor data can be identified based on a determination whether the user is performing a workout session.

In some implementations, determining the asymmetry of the gait of the user can include determining a plurality of steps taken by the user, grouping pairs of steps into respective strides, and determining the asymmetry of the gait of the user for each stride.

In some implementations, determining the asymmetry of the gait of the user for each stride can include determining a respective asymmetry score based on a logistic regression.

In some implementations, the computing device can include the one or more accelerometers and the one or more gyroscopes.

In some implementations, the computing device can be positioned asymmetrically about a center plane of the user.

Other implementations are directed to systems, devices and non-transitory, computer-readable mediums for performing one or more of the techniques described herein.

Particular implementations provide at least the following advantages. In some implementations, the techniques described herein enable computing devices to determine the characteristics of a user's gait more accurately. Based on this information, computing devices can determine the physical health of a patient and monitor the patient's health over time. In some implementations, this also enables computing devices to identity health conditions associated with a user, and in response, take appropriate actions to address those conditions.

The details of one or more embodiments are set forth in the accompanying drawings and the description below. Other features and advantages will be apparent from the description and drawings, and from the claims.

### DESCRIPTION OF DRAWINGS

FIG. 1 is a block diagram of an example mobile device.

FIG. 2A is a diagram showing example potions of a mobile device on a user's body.

FIG. 2B is a diagram showing example directional axes with respect a mobile device.

FIG. 3 is a diagram showing an example acceleration signal with respect to example phases of walking.

FIG. 4 is a diagram showing example frames of references with respect to a mobile device.

FIG. 5 is a diagram showing an example process for estimating an acceleration experienced by a mobile device with respect to a fixed frame of reference

FIG. 6 is a diagram of an example pendulum model.

FIG. 7A is a diagram showing an example process for estimating the walking speed of a user and/or other metrics regarding a gait of the user.

FIG. 7B is a diagram of an example measurement window for estimating the walking speed of a user and/or other metrics regarding a gait of the user.

FIG. 8 is a diagram showing example signals generated using a pendulum model.

FIG. 9 is a diagram of an example processing for determining a symmetry of a user's gait.

FIG. 10 is a diagram of an example process for analyzing the gait of a user.

FIG. 11 is a diagram of another example process for estimating the walking speed of a user and/or other metrics regarding a gait of the user.

FIG. 12 is a flow chart diagram of an example process for electronically monitoring a user's health by analyzing the user's gait.

### DETAILED DESCRIPTION

Example Mobile Device

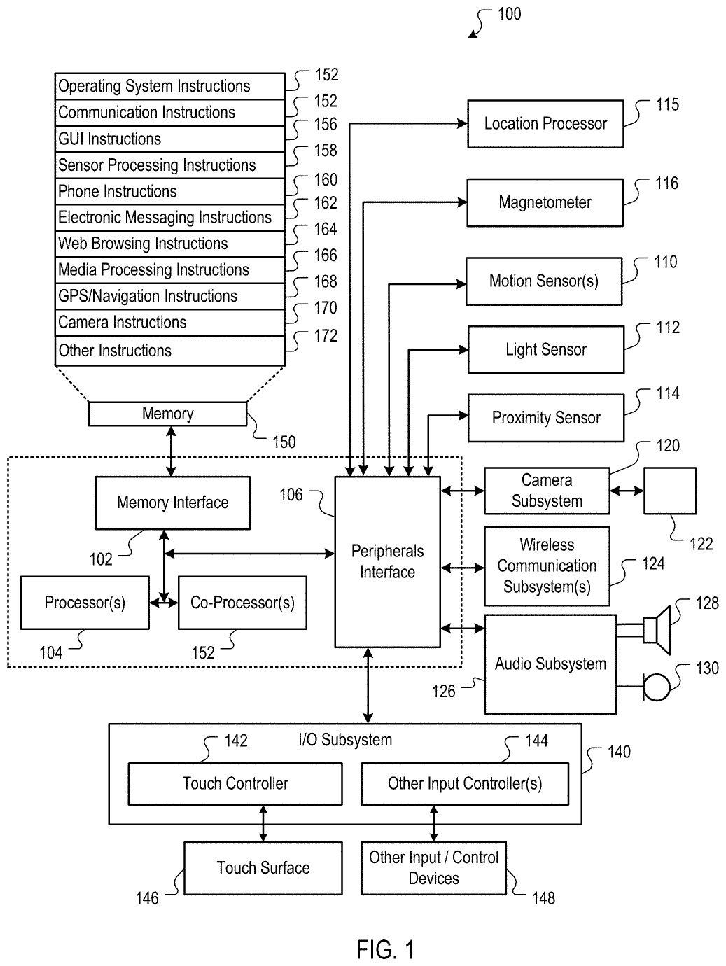

FIG. 1 is a block diagram of an example electronic mobile device 100. In practice, the mobile device 100 can be any portable electronic device for receiving, processing, and/or transmitting data, including but not limited to cellular phones, smart phones, tablet computers, wearable computers (e.g., watches), and the like.

The mobile device 100 can include a memory interface 102, one or more data processor 104, one or more data co-processors 152, and a peripherals interface 106. The memory interface 102, the processor(s) 104, the co-processor(s) 152, and/or the peripherals interface 106 can be separate components or can be integrated in one or more integrated circuits. One or more communication buses or signal lines may couple the various components.

The processor(s) 104 and/or the co-processor(s) 152 can operate in conjunction to perform the operations described herein. For instance, the processor(s) 104 can include one or more central processing units (CPUs) that are configured to function as the primary computer processors for the mobile device 100. As an example, the processor(s) 104 can be configured to perform generalized data processing tasks of the mobile device 100. Further, at least some of the data processing tasks can be offloaded to the co-processor(s) 152. For example, specialized data processing tasks, such as processing motion data, processing image data, encrypting data, and/or performing certain types of arithmetic operations, can be offloaded to one or more specialized co-processor(s) 152 for handling those tasks. In some implementations, the processor(s) 104 can be relatively more powerful than the co-processor(s) 152 and/or can consume more power than the co-processor(s) 152. This can be useful, for example, as it enables the processor(s) 104 to handle generalized tasks quickly, while also offloading certain other tasks to co-processor(s) 152 that may perform those tasks more efficiency and/or more effectively. In some implementations, a co-processor(s) can include one or more sensors or other components (e.g., as described herein), and can be configured to process data obtained using those sensors or components, and provide the processed data to the processor(s) 104 for further analysis.

Sensors, devices, and subsystems can be coupled to peripherals interface 106 to facilitate multiple functionalities. For example, a motion sensor 110, a light sensor 112, and a proximity sensor 114 can be coupled to the peripherals interface 106 to facilitate orientation, lighting, and proximity functions of the mobile device 100. For example, in some implementations, a light sensor 112 can be utilized to facilitate adjusting the brightness of a touch surface 146. In some implementations, a motion sensor 110 can be utilized to detect movement and orientation of the device. For example, the motion sensor 110 can include one or more accelerometers (e.g., to measure the acceleration experienced by the motion sensor 110 and/or the mobile device 100 over a period of time), and/or one or more compasses or gyros (e.g., to measure the orientation of the motion sensor 110 and/or the mobile device). In some implementations, the measurement information obtained by the motion sensor 110 can be in the form of one or more a time-varying signals (e.g., a time-varying plot of an acceleration and/or an orientation over a period of time). Further, display objects or media may be presented according to a detected orientation (e.g., according to a “portrait” orientation or a “landscape” orientation). In some implementations, the motion sensor 110 can also include one or more pedometers that are configured to detect when a user has taken a step, the number of steps that the user has taken, the rate at which the user takes steps (e.g., a step cadence), and/or any other additional information regarding a user's steps. In some implementations, a motion sensor 110 can be directly integrated into a co-processor 152 configured to processes measurements obtained by the motion sensor 110. For example, a co-processor 152 can include one more accelerometers, compasses, gyroscopes, and/or pedometers, and can be configured to obtain sensor data from each of these sensors, process the sensor data, and transmit the processed data to the processor(s) 104 for further analysis.

Other sensors may also be connected to the peripherals interface 106, such as a temperature sensor, a biometric sensor, or other sensing device, to facilitate related functionalities. Similarly, these other sensors also can be directly integrated into one or more co-processor(s) 152 configured to process measurements obtained from those sensors.

A location processor 115 (e.g., a GNSS receiver chip) can be connected to the peripherals interface 106 to provide geo-referencing. An electronic magnetometer 116 (e.g., an integrated circuit chip) can also be connected to the peripherals interface 106 to provide data that may be used to determine the direction of magnetic North. Thus, the electronic magnetometer 116 can be used as an electronic compass.

A camera subsystem 120 and an optical sensor 122 (e.g., a charged coupled device [CCD] or a complementary metal-oxide semiconductor [CMOS] optical sensor) can be utilized to facilitate camera functions, such as recording photographs and video clips.

Communication functions may be facilitated through one or more communication subsystems 124. The communication subsystem(s) 124 can include one or more wireless and/or wired communication subsystems. For example, wireless communication subsystems can include radio frequency receivers and transmitters and/or optical (e.g., infrared) receivers and transmitters. As another example, wired communication system can include a port device, e.g., a Universal Serial Bus (USB) port or some other wired port connection that can be used to establish a wired connection to other computing devices, such as other communication devices, network access devices, a personal computer, a printer, a display screen, or other processing devices capable of receiving or transmitting data.

The specific design and implementation of the communication subsystem 124 can depend on the communication network(s) or medium(s) over which the mobile device 100 is intended to operate. For example, the mobile device 100 can include wireless communication subsystems designed to operate over a global system for mobile communications (GSM) network, a GPRS network, an enhanced data GSM environment (EDGE) network, 802.x communication networks (e.g., Wi-Fi, Wi-Max), code division multiple access (CDMA) networks, NFC and a Bluetooth™ network. The wireless communication subsystems can also include hosting protocols such that the mobile device 100 can be configured as a base station for other wireless devices. As another example, the communication subsystems may allow the mobile device 100 to synchronize with a host device using one or more protocols, such as, for example, the TCP/IP protocol, HTTP protocol, UDP protocol, and any other known protocol.

An audio subsystem 126 can be coupled to a speaker 128 and one or more microphones 130 to facilitate voice-enabled functions, such as voice recognition, voice replication, digital recording, and telephony functions.

An I/O subsystem 140 can include a touch controller 142 and/or other input controller(s) 144. The touch controller 142 can be coupled to a touch surface 146. The touch surface 146 and the touch controller 142 can, for example, detect contact and movement or break thereof using any of a number of touch sensitivity technologies, including but not limited to capacitive, resistive, infrared, and surface acoustic wave technologies, as well as other proximity sensor arrays or other elements for determining one or more points of contact with the touch surface 146. In one implementation, the touch surface 146 can display virtual or soft buttons and a virtual keyboard, which can be used as an input/output device by the user.

Other input controller(s) 144 can be coupled to other input/control devices 148, such as one or more buttons, rocker switches, thumb-wheel, infrared port, USB port, and/or a pointer device such as a stylus. The one or more buttons (not shown) can include an up/down button for volume control of the speaker 128 and/or the microphone 130.

In some implementations, the mobile device 100 can present recorded audio and/or video files, such as MP3, AAC, and MPEG video files. In some implementations, the mobile device 100 can include the functionality of an MP3 player and may include a pin connector for tethering to other devices. Other input/output and control devices may be used.

A memory interface 102 can be coupled to a memory 150. The memory 150 can include high-speed random access memory or non-volatile memory, such as one or more magnetic disk storage devices, one or more optical storage devices, or flash memory (e.g., NAND, NOR). The memory 150 can store an operating system 152, such as Darwin, RTXC, LINUX, UNIX, OS X, WINDOWS, or an embedded operating system such as VxWorks. The operating system 152 can include instructions for handling basic system services and for performing hardware dependent tasks. In some implementations, the operating system 152 can include a kernel (e.g., UNIX kernel).

The memory 150 can also store communication instructions 154 to facilitate communicating with one or more additional devices, one or more computers or servers, including peer-to-peer communications. The communication instructions 154 can also be used to select an operational mode or communication medium for use by the device, based on a geographic location (obtained by the GPS/Navigation instructions 168) of the device. The memory 150 can include graphical user interface instructions 156 to facilitate graphic user interface processing, including a touch model for interpreting touch inputs and gestures; sensor processing instructions 158 to facilitate sensor-related processing and functions; phone instructions 160 to facilitate phone-related processes and functions; electronic messaging instructions 162 to facilitate electronic-messaging related processes and functions; web browsing instructions 164 to facilitate web browsing-related processes and functions; media processing instructions 166 to facilitate media processing-related processes and functions; GPS/Navigation instructions 168 to facilitate GPS and navigation-related processes; camera instructions 170 to facilitate camera-related processes and functions; and other instructions 172 for performing some or all of the processes described herein.

Each of the above identified instructions and applications can correspond to a set of instructions for performing one or more functions described herein. These instructions need not be implemented as separate software programs, procedures, or modules. The memory 150 can include additional instructions or fewer instructions. Furthermore, various functions of the device may be implemented in hardware and/or in software, including in one or more signal processing and/or application specific integrated circuits (ASICs).

### Example Functionality

The mobile device 100 can be used to determine the characteristics of a user's gait. For example, a user can position the mobile device 100 on his body, and walk for a period of time. As the user is walking, the mobile device 100 can collect sensor data regarding movement of the mobile device 100, an orientation of the mobile device 100, and/or other dynamic properties. Based on this information, the mobile device 100 can estimate the characteristics of a user's gait as he walks. As an example, the mobile device 100 can estimate the periods of time during which both of the user's feet are on the ground (e.g., a “double support” interval) and/or the periods of time during which only one of the user's feet are on the ground (e.g., a “single support interval”). As further examples, the mobile device 100 can estimate the walking speed of a user, a step length of the user, a step period of a user, a turning rate or a user, and/or a symmetry of the user's gait, duration or gait cycles within one or more walking segments, among other characteristics.

Further, the mobile device 100 can also use this information to monitor the physical health of a patient over time. For example, based on the characteristics of the user's gait, the mobile device 100 can estimate a mobility of the user, a physical independence of the user, a disease severity of the user, and/or an injury risk of the user. In some implementations, the mobile device 100 can present this information to the user, for example, to assist the user in caring for himself. In some implementations, the mobile device 100 can present this information to others, for example, to assist them in caring for the user. Further, the mobile device 100 can track changes to the user's physical health over time, such that health trend of a user can be determined.

In some implementations, the mobile device 100 can identity a health condition associated with the user, and in response, take an appropriate action to address that condition. For example, the mobile device 100 can identity a progression of a disease, and notify the user or others if the disease has progressed to a sufficiently severe state. As another example, the mobile device 100 can identity risk factors for particular conditions or disease, and notify the user so that the user can modify his behavior and/or seek medical attention. Further, the mobile device 100 can notify others such that medical treatment can be administered and/or further examination can be performed. In some implementations, the mobile device 100 can be used to track the onset and progression of Parkinson's disease, or other diseases that can affect a user's mobility.

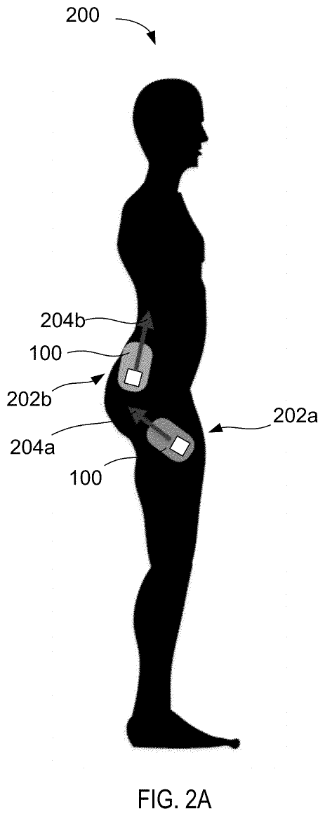

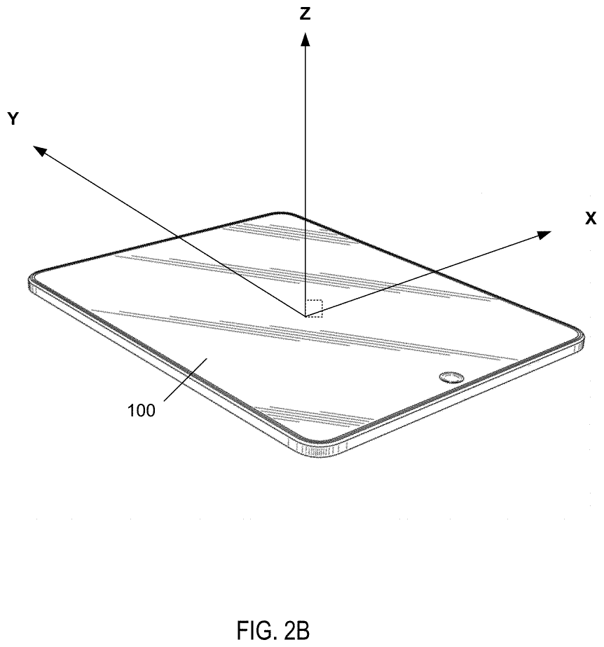

As described above, a user can position the mobile device 100 on his body, and walk for a period of time. FIG. 2A shows two example positions at which a user 200 might position the mobile device 100. As a first example, a user 200 can position a mobile device 100 at a location 202a along his thigh. This could correspond, for example, to the user 200 placing the mobile device 100 in an article of clothing being worn by the user 200, such as in the pocket of a pair of pants, dress, skirt, shorts, jacket, coat, shirt, or other article of clothing. As a second example, a user 200 can position a mobile device 100 at location 202b along his hip. This could correspond, for example, to the user 200 placing the mobile device 100 on a hip-secured support structure, such as a belt clip or hip holster.

Further, the orientation of the mobile device 100 may differ, depend on the location at which is it placed on the user's body. As examples, the orientation 204a of the mobile device 100 at the location 202a and the orientation 204b of the mobile device 100 at the location 202b are shown in FIG. 1. Orientations 204a and 204b can refer, for example, to a vector projecting from a top of the device (e.g., the y-axis shown in FIG. 2B). In some implementations, the mobile device 100 can be positioned asymmetrically on the user's body with respect to the user's left and right directions (e.g., with respect to a center plane, such as a sagittal plane). For example, the mobile device 100 can be positioned closer to a right side of his body than his left side, or vice versa.

As the user walks with the mobile device 100 on his body, the mobile device 100 collects sensor data regarding the motion of the user. For instance, using the motion sensors 110 (e.g., one or more accelerometers), the mobile device 100 can measure an acceleration experienced by the motion sensors 110, and correspondingly, the acceleration experienced by the mobile device 100. Further, using the motion sensors 110 (e.g., one or more compasses or gyroscopes), the mobile device 100 can measure an orientation of the motion sensors 110, and correspondingly, an orientation of the mobile device 100. Further, using the motion sensors 110 (e.g., one or more pedometers), the mobile device 100 can determine the number of steps taken by a user over a period of time and/or the user's step cadence for that period of time. In some implementations, the motion sensors 110 can collect data continuously or periodically over a period of time. In some implementations, the motion sensors 110 can collect motion data with respect to one or more specific directions relative to the orientation of the mobile device 100. For example, the motion sensors 110 can collect sensor data regarding an acceleration of the mobile device 100 with respect to the x-axis (e.g., a vector projecting from a side of the mobile device 100, as shown in FIG. 2B), the y-axis (e.g., a vector projecting from a top of the mobile device 100, as shown in FIG. 2B) and/or the z-axis (e.g., a vector projecting from a front of the mobile device 100, as shown in FIG. 2B), where the x-axis, y-axis, and z-axis refer to a Cartesian coordinate system in a frame of reference of the mobile device 100.

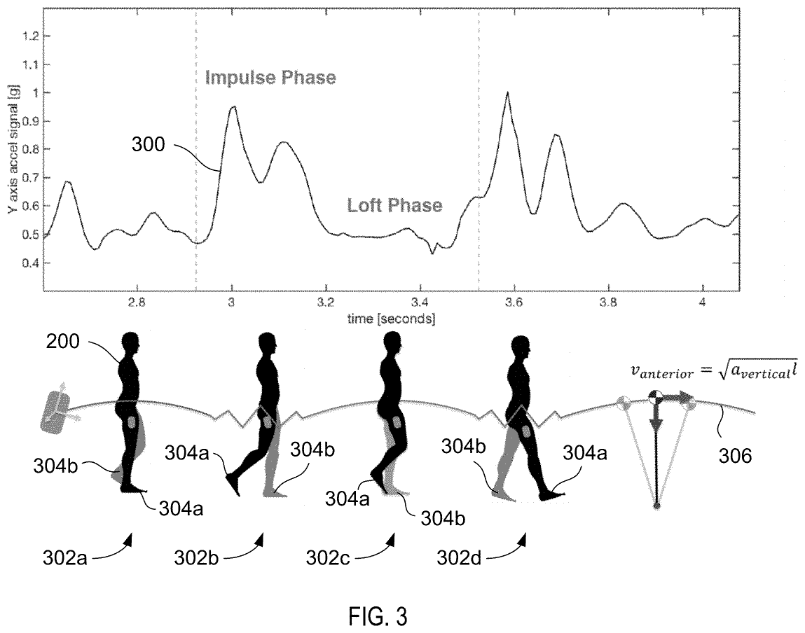

As an example, as shown in FIG. 3, as the user 200 is walking, the mobile device 100 can use the motion sensors 110 to continuously or periodically collect sensor data regarding an acceleration experienced by the motion sensors 110 with respect to y-axis over a period of time. The resulting sensor data can be presented in the form of a time-varying acceleration signal 300.

As the user walks, he alternatingly places a foot on the ground and swings the other in a sequential manner. For example, as shown in FIG. 3, during a first phase 302a, the user 200 positions his right foot 304a on the ground, and swings his left foot 304b in front of the right foot 304a. Thus, only the right foot 304a is in contact with the ground and experiences a stance phase. This first phase 302a—during which only one foot is on the ground—can be referred to as a “single support” interval.

Further, during a second phase 302b, the user 200 contacts the ground with his left foot 304b, while his right foot 304a remains positioned on the ground. Thus, in this phase, both feet 304a and 304b are in contact with the ground. This second phase 302b—during which two feet are on the ground—can be referred to as a “double support” interval.

As the user continues walking, the user repeatedly alternates between single support intervals and double support intervals. For example, during a third phase 302c, the user 200 keeps his left foot 304b on the ground. Meanwhile, the user 200 lifts his right foot 304a off the ground, and swings it in front of the left foot 304b. Thus, only the left foot 304b is in contact with the ground while the right foot 304a experiences a swing phase. This third phase 302c also can be referred to as a loft phase or a single support interval

Further, in a fourth phase 302d, the user 200 contacts the ground with his right foot 304a, while his left foot 304b remains positioned on the ground. Thus, in this phase, both feet 304a and 304b are in contact with the ground. This second phase 302b—during which both feet 304a and 304b are on the ground—also can be referred to as an a double support interval.

Further, as the user walks, he may transition back and forth between a “loft” phase (in which the user is falling with gravity) and an “impulse” phase (in which he is accelerating against gravity). For example, FIG. 3 includes a curve 306 shows the rise and fall of the user over time. Portions of the curve that are falling correspond to the loft phase, and portions of the curve that are rising correspond to the impulse phase.

The acceleration signal 300 varies during each of the loft and impulse phases. For example, as shown in FIG. 3, during the loft phases, the measured acceleration with respect to the y-axis is relatively lower in magnitude (e.g., corresponding to the user falling with gravity). However, during the impulse phases, the measured acceleration with respect to the y-axis increases magnitude (e.g., spikes in magnitude, corresponding to the impact of the user's foot on the ground and the rise of the user against gravity). The mobile device 100 can identify loft and impulse phases, at least in part, based on the acceleration signal 300.

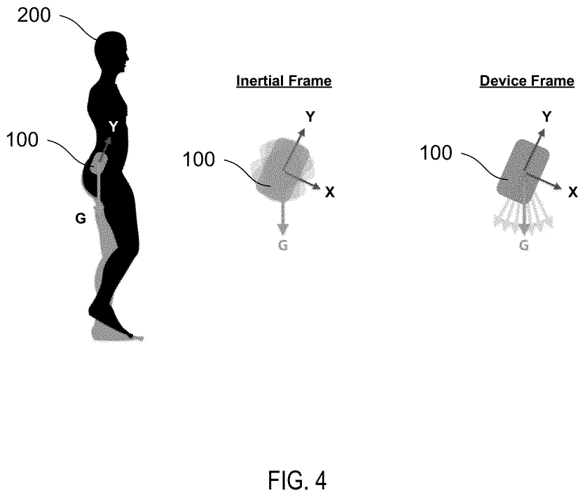

In the example above, the acceleration signal 300 indicates the acceleration experienced by the mobile device 100 with respect to the y-axis of the mobile device. Accordingly, the frame of reference of the acceleration signal 300 depends on the orientation of the mobile device 100 (e.g., a “device frame,” as shown in FIG. 4). In some implementations, the acceleration signal 300 can also indicate the acceleration experienced by the mobile device 100 with respect to multiple different directions. For example, the acceleration signal 300 can include an x-component, a y-component, and a z-component, referring to the acceleration experienced by the mobile device 100 with respect to the x-axis, the y-axis, and the z-axis of the mobile device 100, respectively.

In some implementations, the acceleration signal 300 can be used to estimate an acceleration experienced by the mobile device 100 with respect to a fixed frame of reference (e.g., an “inertial frame” with respect to the direction of gravity, G, as shown in FIG. 4). This can be useful, for example, to obtain a more objective or reproducible representation of the motion of the mobile device 100.

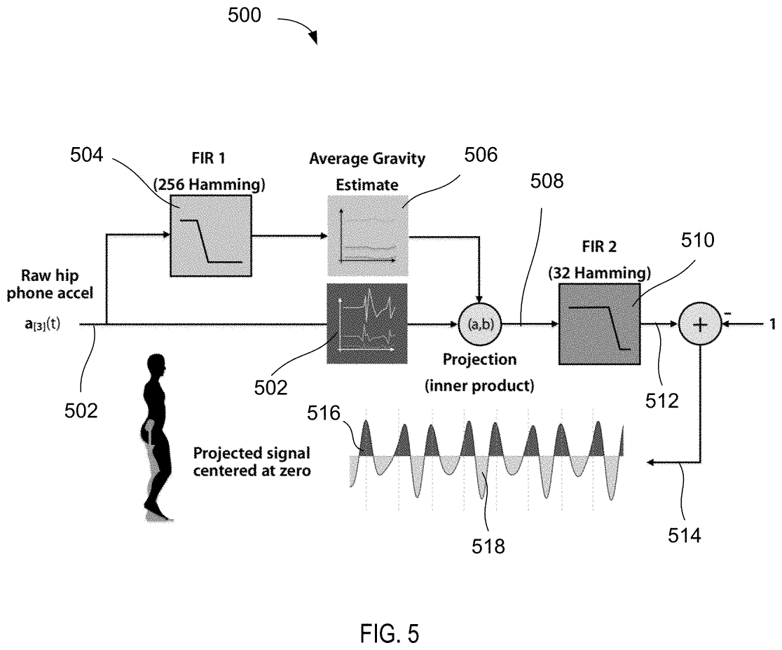

FIG. 5 shows an example process 500 for estimating an acceleration experienced by the mobile device 100 with respect to a fixed frame of reference.

First, the mobile device 100 obtains an acceleration signal 502 indicating the acceleration experienced by the mobile device 100 over a period of time. In this example, the acceleration signal 502 includes three components: an x-component, a y-component, and a z-component, referring to the acceleration experienced by the mobile device 100 with respect to the x-axis, the y-axis, and the z-axis, respectively, in the frame of reference of the mobile device 100. The acceleration signal 502 can be referred to as a “raw” acceleration.

The mobile device 100 filters the acceleration signal 502 using a first low pass filter 504, and obtains a first filtered acceleration signal 506. The first filtered acceleration signal 506 can be used as an estimate for an average gravity with respect to each of the x-axis, y-axis, and z-axis. For example, filtering the acceleration signal 502 can result in a first filtered acceleration signal 506 having an x-component, a y-component, and a z-component, corresponding to an estimate for an average gravity with respect to each of the x-axis, y-axis, and z-axis, respectively. In some implementations, the first low pass filter 504 can be a finite impulse response (FIR) filter. Further, the first low pass filter 504 can filter the acceleration signal 502 according to a window function. As an example, the first low pass filter 504 can filter the acceleration signal 502 according to a Hamming window of width N₁. In practice, the value of N₁ can vary. For example, in some implementations, N₁ can be 256.

Further, the mobile device 100 projects the acceleration signal 502 onto the filtered acceleration signal 506, resulting in a projected acceleration signal 508. This can be performed, for example, by determining an inner product of the acceleration signal 502 and the first filtered acceleration signal 506.

Further, the mobile device 100 filters the projected acceleration signal 508 using a second low pass filter 510, and obtains a second filtered acceleration signal 512. The second filtered acceleration signal 512 can be used as an estimate for the acceleration experienced by the mobile device 100 in the direction of gravity. In some implementations, the second low pass filter 510 can be an FIR filter. Further, the second low pass filter 510 can filter the projected acceleration signal 508 according to a window function. As an example, the second low pass filter 510 can filter the projected acceleration signal 508 according to a Hamming window of width N₂. In practice, the value of N₂ can vary. In some implementations, N₂ can be less than N₁. For example, in some implementations, N₂ can be 32.

In some implementations, the first low pass filter 504 and the second low pass filter 510 can filter signals according to different cut off frequencies. For example, the first low pass filter 504 can have a first cut off frequency f₁, and the second low pass filter 510 can have a different second cut off frequency f₂. In some implementations, f₁ can be less than f₂.

Further, the second filtered acceleration signal 512 can be normalized to remove the effect of gravity. For example, if the acceleration signal indicates acceleration in units g (e.g., where 1 g=32.174 m/s²), 1 g can be subtracted from the second filtered acceleration signal 512 to obtain a normalized acceleration signal 514. As shown in FIG. 5, the normal acceleration signal 514 is a time-varying signal that is “centered” at zero, and includes portions 516 that are greater than zero, and portions 518 that are less than zero.

The portions of the normalized acceleration signal that are greater than zero and the portions of the normalized acceleration signal that are less than zero can be used to estimate the impulse phases and loft phases, respectively, or a user's gait.

Various characteristics of a user's gait can be determined based on the normalized acceleration signal. As an example, the normalized acceleration signal can be used to determine a ratio between the length of time of the impulse phases of the user's gait and the length of time of the loft phases of the user's gait. For instance, a greater ratio could indicate that the user spends more time with both feet on the ground during walking, whereas a smaller ratio could indicate that the user spends more time with a single foot on the ground during walking.

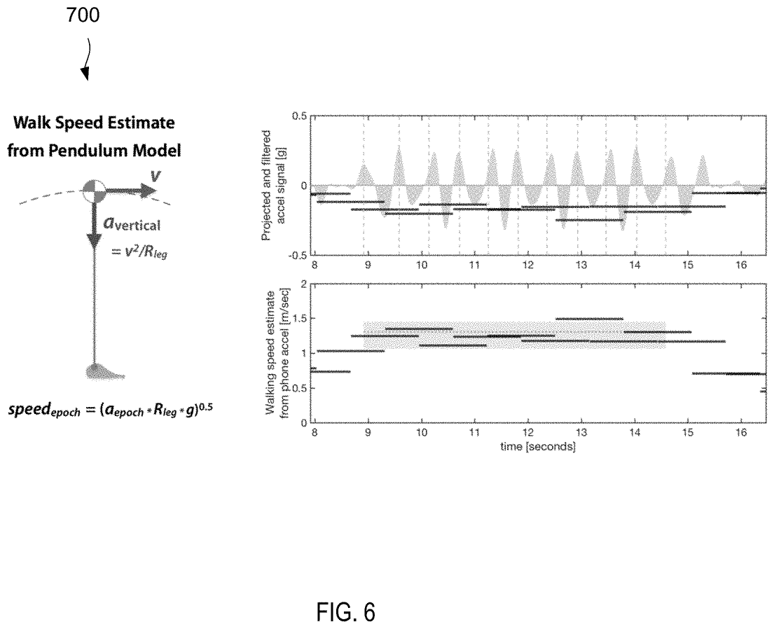

As another example, the normalized acceleration signal can be used to determine a walking speed of the user (e.g., a speed of the user with respect to the ground). For instance, as shown in FIG. 6, the walking speed of the user can be determined using a pendulum model 600. In the pendulum model 600, the user is represented as a swinging pendulum of length R_{leg}, referring to the length of the user's leg. In some implementations, R_{leg} can be determined by measuring the length of the user's leg. In some implementations, R_{leg} can be empirically estimated (e.g., by obtaining the user's height, and estimating the length of the user's leg based on height and leg length data collected from a sample population). The walking speed of the user in a sample epoch, speed_{epoch}, can be estimated using the relationship:

speed_{epoch}=(a_{epoch}*R_{leg}*g)^{0.5},

where a_{epoch} is the normalized acceleration signal during the sample epoch, and g is the acceleration of gravity.

As another example, the normalized acceleration signal can be used to determine a step length of the user (e.g., the length that the user traverses with each step of his gait). For instance, the step length of the user also can be determined using the pendulum model 600. The step length of the user in a sample epoch, step_length_{epoch}, can be estimated using the relationship:

step_length_{epoch}=(a_{epoch}*R_{leg}*g)^{0.5}/step_{cadence},

where a_{epoch} is the normalized acceleration signal during the sample epoch, R_{leg} is the length of the user's leg according to the pendulum model 600, g is the acceleration of gravity, and step_{cadence} is the cadence of the user's gait (e.g., the frequency at which the user places his feet on the ground as he walks).

The step length of the user in a sample epoch, step_length_{epoch}, also can be estimated using the relationship:

step_length_{epoch}=(a_{epoch}*R_{leg}*g)^{0.5}*step_{period},

where step_{period} is the period of the user's gait (e.g., the time period between the user's steps as he walks).

In some implementations, a user can walk multiple times through a particular course (e.g., walk multiple “laps” on a track). As the user walks, the user's average walking speed can be determined for each lap. Further, the average walking speeds for each lap can be compared to determine trends in the user's gait. For example, a determination can be made that the user is slowing down over time, or that the user is speeding up over time. In some implementations, a user can walk multiple different times during a day (e.g., multiple different walking sessions). As the user walks, the user's average walking speed can be determined for each session. Further, the average walking speeds for each of the sessions can be compared to determine trends in the user's gait. For example, a determination can be made that the user is slowing down over time, or that the user is speeding up over time.

As another example, the normalized acceleration signal can be used to determine a Froude Number describing the user's gait. A Froude Number is a dimensionless number defined as the ratio of a flow inertia to an external field (e.g., gravity). The Froude Number also can be determined using the pendulum model 600. For instance, the Froude Number describing the user's gait, Fr, can be estimated using the relationship:

$$F r = \frac{{v}^{2}}{g {R}_{l e g}} ,$$

where v is the velocity of the user, g is the acceleration of gravity, and R_{leg} is the length of the user's leg according to the pendulum model 700.

The mobile device 100 can monitor a user's health using one or more of the characteristics above. For example, as the user walks, the mobile device 100 can monitor (i) a ratio between the length of time of the impulse phases of the user's gait and the length of time of the loft phases of the user's gait, (ii) a walking speed of the user, (iii) a step length of the user, (iv) a Froude Number describing the user's gait, and/or (v) a user's turn rate. Using these characteristics, the mobile device 100 can estimate a physical health of the user. For example, certain values or combinations of values could indicate that a user is relatively healthier, whereas other values or combinations of values could indicate that a user is relatively less healthy. As another example, certain values or combinations of values could indicate an onset and/or severity of a particular disease (e.g., Parkinson's disease), whereas other values or combinations of values could indicate the absence of the disease.

In some implementations, the mobile device 100 can make a determination regarding a user's health based on sample data collected from a sample population. For example, the mobile device 100 can obtain information regarding the gait characteristics of multiple individuals from a sample population, and information regarding a health state of each of those individuals. For instance, the mobile device 100 can obtain, for each individual of the sample population, (i) a ratio between the length of time of the impulse phases of the user's gait and the length of time of the loft phases of the user's gait, (ii) a walking speed of the user, (iii) a step length of the user, (iv) a Froude Number describing the user's gait, and/or (v) a user's turn rate. Further, the mobile device 100 can obtain, for each individual of the sample population, information describing a health of the individual (e.g., a general state of health of the individual, the onset and/or severity of diseases of the individual, a medical history of the individual, and so forth). Further, the mobile device 100 can obtain, for each individual of the sample population, demographic data regarding the individual (e.g., age, height, weight, location, etc.). This information can be obtained, for example, from an electronic database made available to the mobile device 100. In some implementations, the information can be anonymized, such that an individual's health information cannot be attributed to the individual by others.

Using this information, one or more correlations can be identified between the characteristics of a user's gait and the health state of the user. For example, based on the sample data collected from the sample population, a correlation can be identified between one or more particular characteristics of an individual's gait, a particular demographic of the individual, and a generally positive health state of the individual. Accordingly, if the mobile device 100 determines that the user's gait shares similar characteristics and that the user is part of a similar demographic, the mobile device 100 can determine that the user has a generally positive health state. As another example, based on the sample data collected from the sample population, a correlation can be identified between one or more particular characteristics of an individual's gait, a particular demographic of the individual, and the severity of a particular disease of the individual. Accordingly, if the mobile device 100 determines that the user's gait shares similar characteristics and that the user is part of a similar demographic, the mobile device 100 can determine that the user has the same disease with the same severity.

These correlations can be determined using various techniques. For example, in some implementations, these correlations can be identified through the use of one or more “machine learning” techniques such as decision tree learning, association rule learning, artificial neural networks, deep learning, inductive logic programming, support vector machines clustering, Bayesian networks, reinforcement learning, representation learning, similarity and metric learning, sparse dictionary learning, genetic algorithms, rule-based machine learning, learning classifier systems, among others.

Further, the characteristics of a user's gait can be used to determine additional information regarding a user. As an example, the characteristics of a user's gait can be used to determine whether the user is more likely to be independent (e.g., is physically able to care for himself without the assistance of others) or dependent (e.g., is reliant on the assistance of others). For instance, a user having a relatively higher walking speed may be more likely to be independent, whereas a user having a relatively lower walking speed may be more likely to be dependent.

As an example, the characteristics of a user's gait can be used to determine whether the user may require hospitalization or medical care. For instance, a user having a relatively slower walking speed may be more likely require hospitalization (e.g., to treat an injury or disease), whereas a user having a relatively higher walking speed may be less likely to require hospitalization or medical care.

As another example, the characteristics of a user's gait can be used to determine whether the user is prone to falling. For instance, a user having a relatively slower walking speed may be more prone to falling (and thus may be more likely require physical assistance). In contrast, a user having a relatively higher walking speed may be less prone to falling (and thus may be less likely to require physical assistance).

As another example, the characteristics of a user's gait can be used to determine a discharge location for the user after treatment at a medical facility. For instance, for a user having a relatively slower walking speed, a determination can be made to discharge the user to a skilled nursing facility (SNF), such that the user can be further monitored by caretakers. In contrast, for a user having a relatively higher walking speed, a determination can be made to discharge the user to his home.

As another example, the characteristics of a user's gait can be used to determine a degree of mobility of a user. For instance, depending on the walking speed a user, a determination can be made that the user is relatively immobile or relatively mobile. In some implementations, mobility can be classified according to a number of different categories. For example, mobility categories can include “household” mobility, “limited” mobility, “community” mobility,” or “street crossing” mobility, in increasing degrees of mobility.

Further, the mobile device 100 can also use this information to monitor the physical health of a patient over time. For example, the mobile device 100 can track changes to the user's physical health over time, such that health trend of a user can be determined. In some implementations, if one or more of the characteristics of the user's gait change from their normal or “baseline” values, the mobile device 100 can determine that a health of the user has changed.

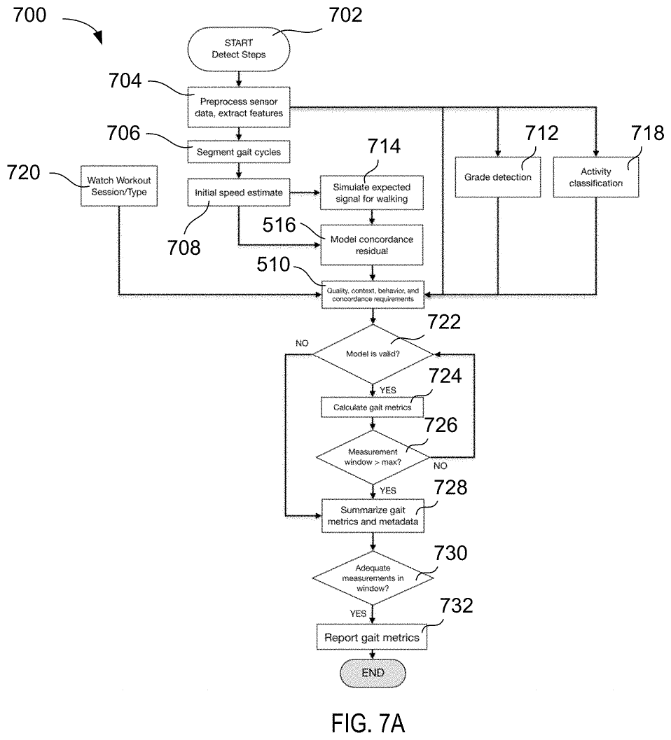

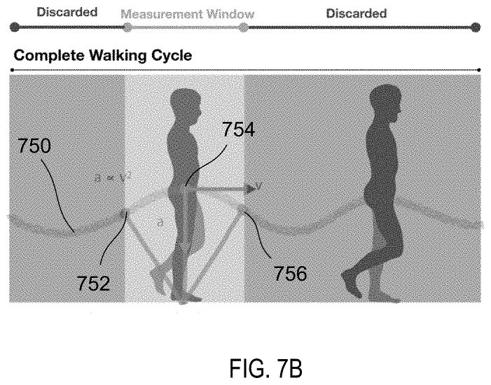

Further, the mobile device 100 can identity a health condition associated with the user, and in response, take an appropriate action to address that condition. For example, the mobile device 100 can identity a progression of a disease, and notify the user or others if the disease has progressed to a sufficiently severe state. For instance, the mobile device 100 can display a notification to the user to inform the user of his health state. Further, the mobile device 100 can transmit a notification to a remote device to inform others of the user's health state (e.g., transmit a message to an emergency response system, a computer system associated with medical personnel, a computer system associated with a caretaker of the user, etc.) As another example, the mobile device 100 can identity risk factors for particular conditions or disease, and notify the user or others so that medical treatment can be administered and/or further examination can be performed. For instance, the mobile device 100 can display a notification to the user to inform the user of his health risks and/or to a remote device to inform others of the user's health risks such that appropriate action can be taken. Notifications can include, for example, auditory information (e.g., sounds), textual information, graphical information (e.g., images, colors, patterns, etc.), and/or tactile or haptic information (e.g., vibrations). As described above, the mobile device 100 can be used to estimate the walking speed of a user and/or other metrics regarding a user's gait. Another example estimation process 700 is shown in FIG. 7A.

According to the process 700, the mobile device 100 determines that the user has taken one or more steps (step 702). For instance, the mobile device 100 can be positioned on the body of the user and obtain sensor data regarding the movement of the user using one or more motions sensors 110 (e.g., one or more accelerometers and/or gyroscopes). The mobile device 100 can determine that the user has taken one or more steps based the characteristics of the sensor data (e.g., by identifying one or more peaks in an acceleration signal indicative of a user indicative of a user taking a step).

Upon determining that the user has taken one or more steps, the mobile device 100 collects additional sensor data regarding the movement of the user over a period of time, and pre-processes the sensor data to extract one or more features from the data (step 704). As an example, the mobile device 100 can collect acceleration data (e.g., indicating a movement of the mobile device, and correspondingly, the movement of the user) and gyroscope data (e.g., indicating an orientation of the mobile device, and correspondingly, the orientation of a portion of the user's body on which the mobile device is being worn). These “raw” sensor measurements can be pre-processed to remove spurious data and/or to improve the consistency of the data. As examples, sensor measurements can be pre-processed to remove signal components from certain ranges of frequencies that are not used to determine the walking speed of a user (e.g., using one or more filters) and/or to frame the sensor measurements with respect to a particular fixed frame of reference.

Further, the mobile device 100 segments the sensor data into more or more portions according to the gait cycles of the user (step 706). For example, the mobile device 100 can segment the sensor data into different portions based on whether each portion of sensor data corresponds to a loft phase of the user's gait or an impulse phase of the user's gait. As another example, the mobile device can segment the sensor data into different portions based on whether each portions of the sensor data corresponds to a single support interval of the user's gait or a double support interval of the user's gait.

Further, the mobile device determines the walking speed of the user based on the segmented sensor data (step 708). Example techniques for determining the user's walking speed are described in further detail below.

The sensor data is also filtered, such that only certain portions of the sensor data that meets certain criteria or requirements are used to determine the walking speed of the user (step 710).

As an example, the mobile device can filter the sensor data based on a detected grade of the surface on which the using is walking (step 712). For instance, the mobile device 100 can include one or more barometers operable to measure an altitude or relative altitude of the mobile device 100. As the user walks, the mobile device 100 can determine a change in altitude of the mobile device 100 over time, and estimate the grade or slope of the surface on which the user is walking. In some implementations, the mobile device 100 can filter the sensor data such that sensor data that was collected when the user was walking on a surface having a level or substantially level grade (e.g., ±1° from level, ±5° from level, ±10° from level, or some other angle from level) is retained, and sensor data that was collected when the user was walking on an inclined surface (e.g., greater than ±1° from level, ±5° from level, ±10° from level, or some other angle from level) is discarded. This can be useful, for example, in improving the accuracy of the measurements and the consistency of measurements between different measurement sessions (e.g., by using only the sensor data that was collected when the user is walking on a level surface).

As another example, the mobile device 100 can simulate sensor data that is expected to be collected by the mobile device 100 as a user walks (step 714). The simulated sensor data can be, for example, one or more signals indicative of “typical” or “ideal” sensor measurements that can be used to estimate the walking speed of a user accurately and consistently. The mobile device 100 can compare the collected sensor data to the simulated sensor data, and based on the comparison, determine whether the collected sensor data can be used to provide sufficiently high-quality results. For instance, the mobile device 100 can determine a residual between the collected sensor data and the simulated sensor data (e.g., indicative of a concordance of the collected sensor data with the simulated sensor data) (step 716). If the collected sensor data has similar characteristics as the simulated sensor data (e.g., the residual is lower than a particular threshold level), the mobile device 100 can determine that the collected sensor data is suitable for use, and can retain the collected sensor data. However, if the collected sensor data has characteristics that are substantially different from those of the simulated sensor data (e.g., the residual exceeds a particular threshold level), the mobile device 100 can determine that the collected sensor data is unsuitable for use, and can discard the collected sensor data. This can be useful, for example, in improving the accuracy and the consistency of measurements between different measurement sessions (e.g., by using only the collected sensor data that is of sufficiently high quality).

As another example, the mobile device 100 can filter the sensor data based on the type of activity that the user was performing at the time that the sensor data was collected (step 718). For instance, the mobile device 100 can include an activity classifier that determines a type of activity that is being performed by a user at any given time (e.g., walking, jogging, running, swimming, sitting, biking, etc.). As an example, the activity classifier can determine the type of activity that is being performed based sensor data collected by the mobile device 100 (e.g., by identifying patterns of sensor data indicative of certain types of activities, such as certain patterns of movements) and/or based on input from the user (e.g., manual input indicating the current activity that is being performed by the user). The mobile device 100 can filter the collected sensor data such that sensor data that was collected when the user was performing a certain type of activity (e.g., walking) is retained, and sensor data that was collected when the user was performing other types of activities (e.g., jogging, running, swimming, sitting, biking, etc.) is discarded. This can be useful, for example, in improving the accuracy and the consistency of measurements between different measurement sessions (e.g., by using only the sensor data that was collected during a specific type of activity).

As another example, the mobile device 100 can filter the sensor data based on whether the user is engaging in a workout session (e.g., a dedicated exercise routine) and/or the type of workout that the user is engaging in at the time that the sensor data was collected (step 720). For example, the user may be running a particular application on the mobile device 100 that guides him in his workout (e.g., an exercise training application that instructs the user to perform certain activities as a part of the workout). The mobile device 100 can determine, based on information provided by the application, whether the user is engaging in a workout session and/or the type of workout that the user is engaging in. The mobile device 100 can filter the collected sensor data such that sensor data that was collected when the user was engaged a workout session and/or a performing a particular type of workout is retained, and sensor data that was collected when the user was not engaged a workout session and/or was performing another type of workout is discarded. This can be useful, for example, in improving the accuracy and the consistency of measurements between different measurement sessions (e.g., by using only the sensor data that was collected during a workout session and/or a specific type of workout).

Further, the mobile device 100 determines whether a physics-based model is applicable to the filtered sensor data (step 722). As an example, the mobile device 100 can use the physics-based pendulum model shown and described with respect to FIG. 6. If the filtered sensor data conforms to that model (e.g., the sensor data can be approximated accurately using the model), the mobile device 100 can use the model to calculate the walking speed of the user and/or other metrics regarding the user's gait using sensor data collected within a particular measurement window (e.g., as described with respect to FIG. 6) (step 724). As shown in FIG. 7B, in some implementations, the pendulum model can represent the movement of a user's leg according to a sinusoidal or approximately sinusoidal pattern 750 (e.g., corresponding to the swinging movement of the top of one of the user's legs when the bottom of that leg is in contact with the ground). The measurement window can correspond to the interval of the sinusoid pattern beginning from a first inflection point 752 of the sinusoidal pattern, extending through the crest 754 of the sinusoidal pattern, and ending at a second inflection point 756 of the sinusoidal pattern. Sensor falling outside of the measurement window can be discarded.

The mobile device 100 continuously uses the model to calculate the walking speed of the user and/or other metrics regarding the user's gait using sensor data until the end of the measurement window (step 726). After the end of the measurement window, the mobile device summarizes the walking speed of the user and/or other metrics regarding the user's gait during the measurement window (step 728).

Alternatively, if the mobile device 100 determines that the physics-based model is not applicable to the filtered sensor data (e.g., the sensor data cannot be approximated accurately using the model), the mobile device 100 refrains from using the model to calculate the walking speed of the user and/or the metrics regarding the user's gait during the measurement window.

Further, the mobile device 100 determines whether adequate measurements have been obtained in the measurement window (step 730). For example, the mobile device 100 can determine whether sensor data was collected over a sufficiently long period of time (e.g., greater than a threshold amount of time) and/or whether sensor data was collected over a sufficiently long walking distance (e.g., greater than a threshold distance). These thresholds can be determined empirically (e.g., by a developer of the mobile devise 100 based on experimental data).

Upon determining that adequate measurements have been obtained, the mobile device 100 determines the walking speed of the user and/or other metrics regarding the user's gait that were measured over the measurement window, and presents the measurements to a user for review (step 732). In some implementations, the mobile device can also determine a measurement quality metric associated with the measurement (e.g., indicating an estimated reliably and/or accuracy of the measurement).

In some implementations, a mobile device 100 can determine a symmetry of the user's gait. For example, the mobile device 100 can determine, based on sensor data, whether the user is favoring one leg over other while walking, and if so, the degree of which is he favoring that leg. For example, the mobile device 100 can determine, based on sensor data, whether the user is moving one leg differently than the other, and if so, the degree of difference between the two.

The degree of symmetry (or asymmetry) of a user's gait can be expressed using one or more metrics. As an example, one metric of symmetry is the user's swing symmetry. The user's swing symmetry refers to the ratio between (i) the period of time during which the user's “affected” leg (e.g., a leg that is physically impaired or otherwise restricted, such as by a leg or knee brace) is off the ground during a step cycle (e.g., the period of time that the user's affected leg is swinging) and (ii) the period of time during which the user's “unaffected” leg (e.g., a leg that is not physically impaired or otherwise restricted) is off the ground during a step cycle (e.g., the period of time that the user's unaffected leg is swinging)

As another example, another metric of symmetry is the user's stance symmetry. The user's stance symmetry refers to the ratio between (i) the period of time during which the user's “affected” leg is on the ground during a step cycle (e.g., the period of time that the user's affected leg is on the ground) and (ii) the period of time during which the user's “unaffected” leg is on the ground during a step cycle (e.g., the period of time that the user's unaffected leg is on the ground).

As another example, another metric of symmetry is the user's overall symmetry. The user's overall symmetry refers to the ratio between (i) the user's swing-stance symmetry for the “affected” leg and (ii) the user's swing-stance symmetry for the user's “unaffected” leg. The swing-stance symmetry for the “affected” leg is the period of time during which the user's “affected” leg is off the ground during a step cycle, divided by the period of time during which the user's “affected” leg is on the ground during a step cycle. The swing-stance symmetry for the “unaffected” leg is the period of time during which the user's “affected” leg is off the ground during a step cycle, divided by the period of time during which the user's “affected” leg is on the ground during a step cycle.

In some implementations, the degree of symmetry of user's gait can be classified into one or more categories based on one or more of these metrics. As an example, if the user's overall symmetry is between 0.9 and 1.1, the user's gait can be classified as “normal” (e.g., indicating that the user's gait is substantially symmetrical). As another example, if the user's overall symmetry is between 1.1 and 1.5, the user's gait can be classified as “mildly asymmetric.” As another example, if the user's overall symmetry is greater than 1.5, the user's gait can be classified as “severely asymmetric.” Although example categories and threshold values are described above, other categories and/or threshold values are also possible, depending on the implementation. In some implementations, categories and their corresponding threshold values can be selected empirically (e.g., based on experiments performed on a sample population).

In some implementations, the degree of symmetry of user's gait is determined by observing the movement of both of the user's legs (e.g., using a pressure sensitive step mat). However, in some implementations, the degree of symmetry of a user's gait can be determined using a single mobile device 100 positioned on a single point on the user's body (e.g., on the user's hip or on the user's thigh) using one or more of the techniques described herein.

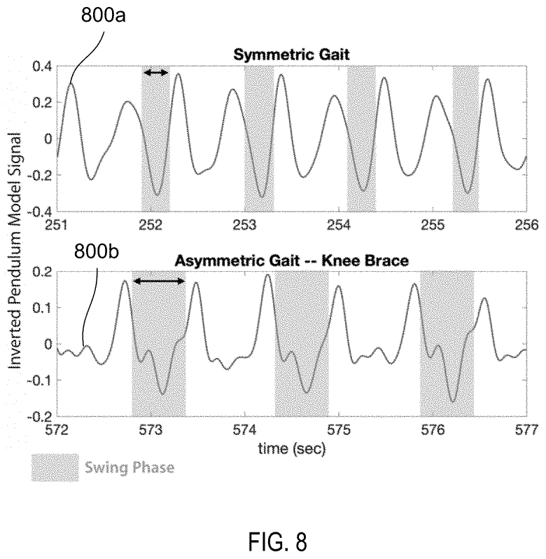

As an example, FIG. 8 shows two signals 800a and 800b generated using a pendulum model (e.g., as shown and described with respect to FIG. 6). In this example, the signal 800a was generated based on sensor data obtained from a user having a symmetric gait, and the signal 800b was generated based on sensor data obtained from a user having an asymmetric gait (e.g., a user wearing a knee brace on one leg). As shown in FIG. 8, of the two signals, the signal 800a more closely resembles a sinusoidal pattern, indicating that the user is swinging and setting each of his legs in a substantially similar manner. In contrast, the signal 800b is more irregular (e.g., having one or more inflection changes between neighboring crests and troughs), indicating that the user is swinging and/or setting his each of his legs in a different manner. As an example, during each swing phase of one of the first user's legs (indicated by the shaded interval), the signal 800a has a single local minimum, a smooth increasing transition to the local minimum, and a smooth decreasing transition from the local minimum. In contrast, during each swing phase of one of the second user's legs (indicated by the shaded interval), the signal 800b has a multiple local minima, and an irregular or erratic transition to and from each minima. Accordingly, the degree of symmetry of a user's gait can be ascertained, at least in part, by modeling a user's gait using a pendulum model, and determining the degree to which the modeled signal approximates a sinusoidal pattern.

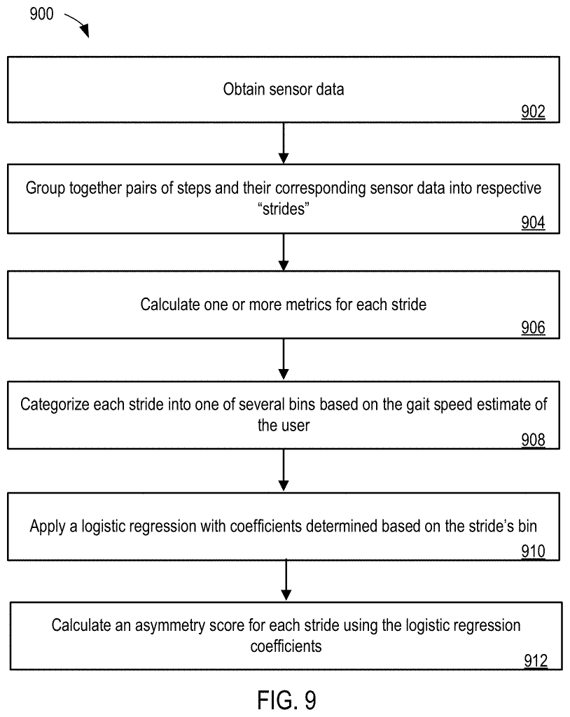

In some implementations, the degree of symmetry of user's gait can be determined algorithmically based on one or more input parameters. An example process 900 for determining the symmetry of a user's gait is shown in FIG. 9.

According to the process 900, a mobile device 100 obtains sensor data regarding multiple steps taken by the user over a period of time (step 902). In some implementations, the mobile device 100 can be positioned on a user's body (e.g., on the user's hip or thigh).

Further, the mobile device 100 groups together pairs of steps (and their corresponding sensor data) into respective “strides” (step 902). As an example, a stride can to defined as the period of time in which a particular leg is on the ground (e.g., a “stance loft”) followed by a period of time in which the leg is off the ground (e.g., a “swing loft”), where there is less than a threshold amount of time (e.g., 1 second) between the end of the stance left and the beginning of the next stance loft.

Further, the mobile device 100 calculates one or more metrics for each stride (step 906), such as using the pendulum model shown and described with respect to FIG. 6. As an example, the mobile device 100 can calculate metrics such as the average step speed of a user during different phrases of his gait, an orientation of the mobile device during different phrases of the user's gait, the amount of time that the user is in each of the different phrases of his gait, and/or any other characteristics of the user's gait.

Further, each stride is categorized into one of several bins based on the gait speed estimate of the user (step 908). Further, different gait models can be used to analyze the gait of the user, depending on the gait speed estimate. For example, a first gait model can be used if the user has a relatively faster gait speed, whereas a second gait model can be used if the user has a relatively slower gait speed. This can be beneficial, for example, as the characteristics of a user's gait may differ, depending on the speed of his gait (e.g., the user's jogging gait may be different than the user's walking gait). In some implementations, a user's strides can be categorized on a continuous basis (e.g., as a continuous variable input). In some implementation a user's strides can be coarsely binned over time (e.g., by binning the strides to different sets of coefficients for slow, moderate, or fast walking in any number of walking segments).

Further, for each stride, a logistic regression is applied with coefficients determined based on the stride's bin (step 910). For example, a linear relationship can be determined between each of the calculated metrics and the user's walking speed. Further, in the linear relationship, each metric can be weighted by a respective linear coefficient. The linear coefficients can be calculated using a logistic regression (e.g., by identifying the linear coefficients that result in a sufficiently accurate calculation of the user's walking speed, given particular ranges of coefficient values). Further, different linear coefficients can be used for each of the different bins. An asymmetry score (e.g., representing the degree of asymmetry of the user's gait) is calculated for each stride using the logistic regression coefficients (step 912).

In some implementations, a particular stride can be classified as asymmetric if its corresponding asymmetry score is above a threshold value (e.g., 0.5). In some implementations, when classifying a group of strides (e.g., a bout, a lap, or other group), the group of strides can be classified as asymmetric if the mean of the asymmetry scores for the strides in the group is above a threshold value (e.g., 0.5). In some implementations, when classifying a group of strides, the group of strides can be classified as asymmetric if a certain percentage of the strides in the group are individually classified as asymmetric.

Although example threshold values are described above, in practice, other threshold values are also possible, depending on the implementation (e.g., 0.1, 0.2, 0.3., 0.4, 0.5, 0.6, 0.7, 0.8, 0.9, or any other value). In some implementations, threshold values can be determined empirically (e.g., based on experiments conducted on a sample population).

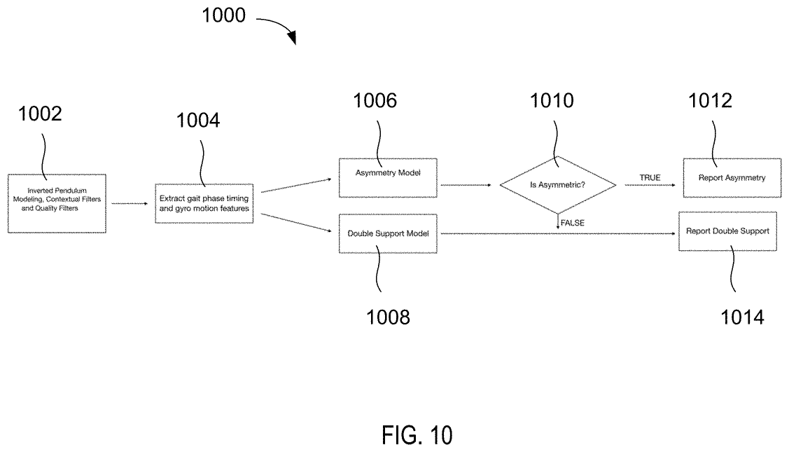

In some implementations, a mobile device 100 can selectively apply an asymmetry model and/or a double support model to analyze the gait of a user, depending on the characteristics of the gait. As an example, FIG. 10 shows an example process 1000 for analyzing the gait of a user.

According to the process 1000, the mobile device 100 obtains sensor data regarding the movement of a user as he walks, models the movement of a user's leg using a pendulum model based on the sensor data, and applies one or more contextual or quality filters to the sensor data (step 1002). As an example, the mobile device 100 can perform some or all of the process 700 shown in FIG. 7A.

Based on the filtered sensor data and the pendulum model, the mobile device 100 extracts information regarding the user's gait (step 1004). For example, the mobile device 100 can determine the timing of each of the phases of the user's gait (e.g., swing phases and stance phases). Further, the mobile device 100 can determine the orientation (or changes in the orientation) of the mobile device over time using sensor data obtained from one or more gyroscopes.

The mobile device 100 can analyze the gait of the user using an asymmetry model (step 1006) and/or a double support model (step 1008), as described herein. Example asymmetry models are described above. For instance, an asymmetry model can be performed using a logistic regression technique, as described above with respect to FIG. 9. Example double support models are described above.

In some implementations, based on the asymmetry model, the mobile device 100 can determine whether the user's gait is asymmetric (step 1010). If so, the mobile device 100 can report the asymmetry and the degree of asymmetry to the user (step 1012). Alternatively, if not, the mobile device 100 can refrain from reporting an asymmetry to the user.

In some implementations, based on the double support mode, the mobile device 100 can determine information regarding the user's gait and/or physical health, and report the information to the user (step 1014). For example, the mobile device can determine one more characteristics of the user's gait, such as the user's walking speed, step length, turning speed, among others, and report one or more of those characteristics to the user. Further, the mobile device 100 can determine the user's physical health, and onset of a diseased, and/or a severity of a disease, and report this information to the user.

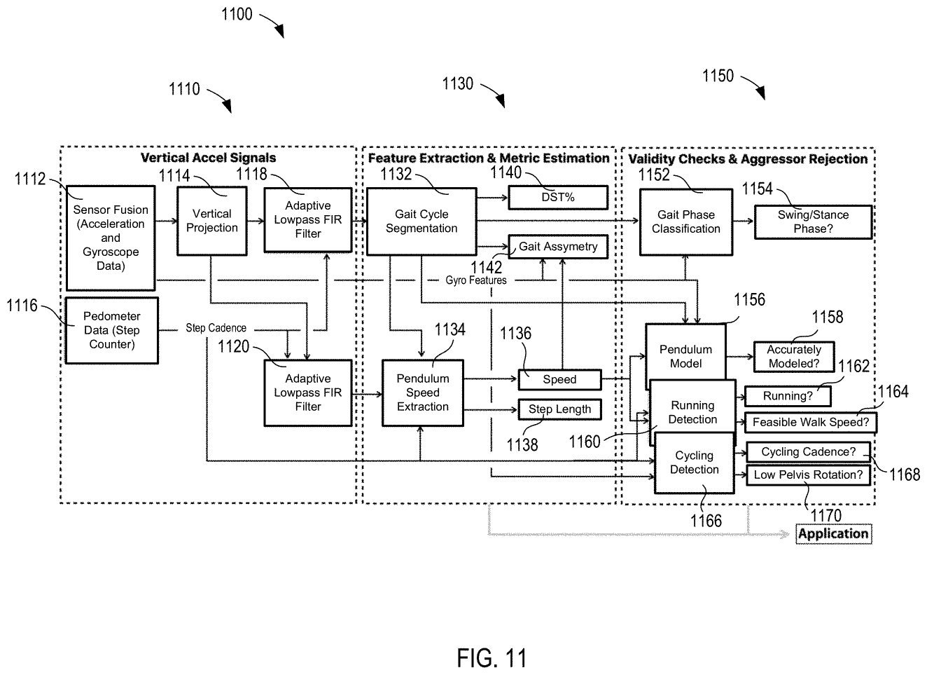

FIG. 11 shows another example process 1100 for estimating the walking speed of a user and/or other metrics regarding a gait of the user. In some implementations, the process 1100 can be performed, at least in part, by a mobile device 100 that is positioned on a user's body.

In general, the process 1100 includes determining acceleration signals representing motion in a vertical direction with respect to a fixed frame of reference (e.g., an “inertial frame” with respect to the direction of gravity) (block 1110), extracting features and estimating metrics based on the vertical acceleration signals (block 1130), and performing validity checks to reduce the occurrence of inaccurate, unreliable, and/or otherwise invalid data (block 1150).

According to the process 1100, a mobile device 100 obtains sensor data from one or more motion sensors 110 (e.g., one or more accelerometers and/or gyroscopes) (sub-block 1112). As an example, the mobile device 100 can collect acceleration data (e.g., indicating a movement of the mobile device, and correspondingly, the movement of the user) and gyroscope data (e.g., indicating an orientation of the mobile device, and correspondingly, the orientation of a portion of the user's body on which the mobile device is being worn). In some implementations, this may be referred to as “sensor fusion” (e.g., obtaining and combining sensor data from multiple types of sensors).

Further, the mobile device 100 determines a vertical projection of the acceleration data (sub-block 1114). As an example, the mobile device 100 can determine the orientation of the mobile device 100 with respect to the inertial frame using the gyroscope data. Further, the mobile device 100 can determine the components of the acceleration data that extend along the vertical direction with respect to the inertial frame (e.g., opposite the direction of gravity). As another example, the mobile device 100 can determine the vertical projection of the acceleration data, at least in part, according to the process 500 (e.g., as described with reference to FIG. 5).

Further, the mobile device 100 obtains pedometer data regarding the steps taken by the user (sub-block 1116). As an example, the mobile device 100 can determine when a user has taken each step. Further, the mobile device 100 can determine the number of steps that the user has taken over a period of time (e.g., a step counter), and determine the rate at which the user takes steps over the period of time (e.g., a step cadence).

Further the mobile device 100 filters the vertically projected acceleration data according to an adaptive low pass finite impulse response (FIR) filter (sub-block 1118). The filtering parameters of the adaptive low pass FIR filter 1118 can be dynamically adjusted based on the step cadence of the user. For example, the filtering parameters of the adaptive low pass FIR filter 1118 can be selected to maintain a consistent number of harmonics (e.g., frequencies that are integer multiples of a particular fundamental frequency) of the vertically projected acceleration data in the pass band of the filter 1118. In some implementations, the adaptive low pass FIR filter 1118 can filter the vertically projected acceleration according to a window function (e.g., according to a window having a particular width or time duration).

Further, the vertically projected acceleration data can be filtered using another adaptive low pass FIR filter (sub-block 1120). As described above, the filtering parameters of the adaptive low pass FIR filter can be dynamically adjusted based on the step cadence of the user. For example, the filtering parameters of the adaptive low pass FIR filter can be selected to maintain a consistent number of harmonics (e.g., frequencies that are integer multiples of a particular fundamental frequency) of the sensor data in the pass band of the filter. In some implementations, the adaptive low pass FIR filter 1120 can retain information regarding the swing frequency and step frequency of the user's gait, and filter out other spectral information (e.g., other harmonics of the acceleration data). In some implementations, the adaptive low pass FIR filter 1120 can filter the vertically projected acceleration according to a window function (e.g., according to a window having a particular width or time duration).

One or more features and/or metrics regarding the user are determined based on the filtered vertically projected acceleration data (block 1130). For example, the vertically projected acceleration data can be segmented into one or more gait cycles (sub-block 1132). Example techniques for segmenting sensor data (e.g., acceleration data) into gait cycles are described, for instance, with reference to FIG. 7.

Further, the mobile device 100 determines speed metrics regarding the user's gait (sub-block 1134) based on the output of the adaptive low pass FIR filter 1120 (e.g., the filtered, segmented, and vertically projected acceleration data) and/or the output of the pedometer. As an example, the mobile device 100 can determine a walking speed of the user (sub-block 1136). As another example, the mobile device 100 can determine a step length of the user (sub-block 1138). In some implementations, the walking speed and/or step length of the user can be determined using a pendulum model (e.g., as described with reference to FIG. 6).

Further, the mobile device 100 determines additional metrics regarding a user's gait. For example, the mobile device 100 can determine the percentage of time in which the user's gait is in a double support interval (e.g., double support time percentage, or “DST %”) (sub-block 1140). This metric can be determined, at least in part, based on the output of the adaptive low pass FIR filter 1118. Example techniques for determining when the user's gait is in a single support interval or a double support interval are described above.

As another example, the mobile device 100 can determine the symmetry or asymmetry of the user's gait (sub-block 1142). This metric can be determined, at least in part, based on the output of the adaptive low pass FIR filter 1118, gyroscope data, and/or the user's determined walking speed. Example techniques for determining the symmetry or asymmetry of a user's gait are also described above.

Further, the mobile device 100 can perform validity checks to reduce the occurrence of inaccurate, unreliable, and/or otherwise invalid data (block 1150). For example, the mobile device 100 can retain subsets of the metrics and features that are more likely to be accurate and/or reliable (e.g., those that were calculated based on data obtained while the user was walking, moving in a way that can be accurately modeled by a pendulum model, etc.). Further, the mobile device 100 can discard or otherwise ignore subsets of the metrics and features that are less likely to be accurate and/or reliable (e.g., those that were calculated based on data obtained while the user was running or cycling, moving in a way that cannot be accurately modeled by a pendulum model, etc.). In some implementations, discarding or otherwise ignoring certain subsets of the metrics and features may be referred to as “aggressor rejection.”

For example, the mobile device 100 can determine, based on the segmented vertically projected acceleration data and gyroscope data, a gait phase associated with each of the segments (sub-block 1152). In some implementations, the mobile device 100 can determine the gait phase specifically for the side of the user's body on which the mobile device 100 is positioned. For instance, if the mobile device 100 is positioned on the left side of the user's body, the mobile device 100 can determine, for each segment of the vertically projected acceleration data, whether the segment corresponds to a swing phase of the user's left leg (e.g., a phase during which the user's left foot is swinging forward) or a stance phase of the user's left leg (e.g., a phase during which the user's left foot is in contact with the ground (sub-block 1154). In some implementations, the mobile device 100 can discard or otherwise ignore the metrics and features that were determined for segments corresponding to a swing phase, and retain the metrics and features that were determined for segments that do not correspond to a swing phase (e.g., the stance phase). Example techniques for determining the phase of a user's gait are described above (e.g., with reference to FIG. 7B).

As another example, the mobile device 100 can determine, based on the segmented vertically projected acceleration data (e.g., vertically projected acceleration data that is segmented according to a gait phase, as described with reference to sub-block 1132), gyroscope data, and the walking speed of the user, whether the user's gait can be accurately modeled using a pendulum model (sub-block 1158). In some implementations, the mobile device 100 can retain the metrics and features that were determined for segments that can be accurately modeled using the pendulum model, and discard or otherwise ignore metrics and features that were determined for segments that cannot be accurately modeled using the pendulum model. In some implementations, each of the segments can be associated with a confidence metric indicating the likelihood that the segment that can be accurately modeled using the pendulum model. Metrics and features for segments having a confidence metric that exceeds a threshold level can be retained, whereas metrics and features for segments having a confidence metric that does not exceed the threshold level can be discarded or otherwise ignored. Example techniques for modeling a user's gait using a pendulum model are described above (e.g., with reference to FIG. 6).

As another example, the mobile device 100 can determine whether a user is walking (e.g., as opposed to performing some other activity, such as running, cycling, etc.).

For example, the mobile device 100 can determine, based on the determined speed of the user and the step cadence of the user, whether the user is running (sub-blocks 1160 and 1162). For instance, the mobile device can determine that the user is running if the user's speed is greater than a particular threshold speed.

Further, the mobile device 100 can determine that the user's speed is a physically possible walking speed (sub-block 1164). As an example, the mobile device 100 can determine that the user's speed is a physically possible walking speed if the user's speed is less than a particular threshold speed. As an example, the mobile device can determine that the user's speed is a physically possible walking speed based on the user's height. For example, the height of a user may be correlated with the walking speeds of the user (e.g., a taller user may walk more quickly than a shorter user). If a particular user is traveling at a speed that exceeds an expected range (e.g., determined based on the user's height), the mobile device 100 can determine that the user is not walking.

In some implementations, the mobile device 100 can retain the metrics and features that were determined for segments corresponding to the user walking, and discard metrics and features that were determined for segments corresponding to the user running and/or traveling at a speed that is not a physically possible walking speed.

For example, the mobile device 100 can determine, based on the speed of the user and step cadence of the user, whether the user is cycling (sub-block 1166). For example, the mobile device can determine whether the user's step cadence is similar to or concordant with a user taking steps, as opposed to a user continuously swinging his legs (e.g., pedaling a bicycle) (sub-block 1168).

As another example, the mobile device 100 can determine whether the rotation of parts of the user's body (e.g., the user's pelvis) is within a particular physiological range that would be expected if the user is walking (e.g., rather than cycling) (block 1170). For example, if the rotation of the user's pelvis is within a particular range, this may be indicative of the user walking. However, if the rotation of the user's pelvis is not within that range (e.g., the rotation is less than the range), this may be indicative of the user cycling. In some implementations, the mobile device 100 can determine the rotation of the user's pelvis (or any other body part) based on sensor data obtained by one or more motion sensors, such as accelerometers and/or gyroscope).

In some implementations, the mobile device 100 can retain the metrics and features that were determined for segments corresponding to the user walking, and discard metrics and features that were determined for segments corresponding to the user cycling (e.g., segments in which the user's step cadence are concordant with a user continuously swinging his legs and/or segments in which the rotation of the user's pelvis is less than an expected range).

In some implementations, at least some of the data that is collected, generated, and/or processed as part of the process 1100 can be displayed to a user (e.g., using a graphical user interface of an application) and/or stored for future retrieval and processing.

### Example Process

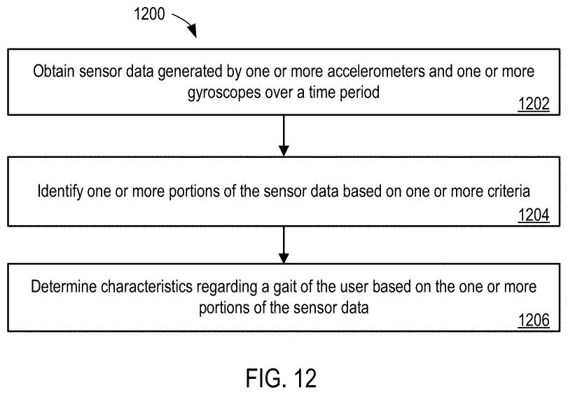

An example process 1200 for electronically monitoring a user's health by analyzing the user's gait is shown in FIG. 12. In some implementations, the process 1100 can be used to determine the characteristics of a user's gait and/or monitor the physical health of a patient over time. The process 1200 can be performed for example, using the system 100 shown in FIG. 1. In some implementations, some or all of the process 1200 can be performed by a co-processor of a computing device. The co-processor can be configured to receive motion data obtained from one or more sensors, process the motion data, and provide the processed motion data to one or more processors of the computing device.

According to the process 1200, a computing device obtains sensor data generated by one or more accelerometers and one or more gyroscopes over a time period (step 1202). The sensor data includes an acceleration signal indicative of an acceleration measured by the one or more accelerometers over a time period. The sensor data also includes an orientation signal indicative of an orientation measured by the one or more gyroscopes over the time period. The one or more accelerometers and the one or more gyroscopes are physically coupled to a user walking along a surface.

The computing device identifies one or more portions of the sensor data based on one or more criteria (step 1204). Techniques for identifying one or more portions the sensor data are described above, for example with respect to FIGS. 7A and 7B.

As an example, the one or more portions of the sensor data can be identified based on an estimated grade of the surface. The grade of the surface can be estimated based on a barometer measurement obtained from a barometric sensor.

As another example, the one or more portions of the sensor data can be identified based on a comparison between the acceleration signal and a simulated acceleration signal. The simulated acceleration signal can be determined based on a pendulum model.

As another example, the one or more portions of the sensor data can be identified based on an estimated activity type of the user during the time period. The one or more portions of the sensor data can be identified based on a determination whether the user is performing a workout session.

The computing device determines characteristics regarding a gait of the user based on the one or more portions of the sensor data. The characteristics include a walking speed of the user and an asymmetry of the gait of the user. Techniques for identifying one or more portions the sensor data are described above, for example with respect to FIG. 3-11.

In some implementations, the asymmetry of the gait of the user can be determined by determining a plurality of steps taken by the user, grouping pairs of steps into respective strides, and determining the asymmetry of the gait of the user for each stride (e.g., as described with respect to FIG. 9). Further, for each stride, a respective asymmetry score can be determined based on a logistic regression.

In some implementations, the characteristics can also include step length of the user and/or a percentage of time that both feet of the user are contacting the ground during a cycle of the gait of the user (e.g., for each gait cycle, the amount of time that the user is in a double support interval, divided by the total time of the gait cycle).

In some implementations, the characteristics regarding a gait of the user can be estimated based on a pendulum model having the acceleration signal as an input. An example pendulum model is described above, for example, with respect to FIG. 6.

In some implementations, the process 1200 can also include determining, based on the sensor data, the acceleration with respect to an inertial frame of reference.

In some implementations, the computing device can include the one or more accelerometers and the one or more gyroscopes. For example, the computing device can be a smart phone or a wearable device (e.g., a smart watch) that includes the one or more accelerometers and the one or more gyroscopes. Further, the computing device can be positioned asymmetrically about a center plane of the user. For example, the computing device can be worn closer to a right side or a left side of the user.

Other Example Implementations

The features described may be implemented in digital electronic circuitry or in computer hardware, firmware, software, or in combinations of them. The features may be implemented in a computer program product tangibly embodied in an information carrier, e.g., in a machine-readable storage device, for execution by a programmable processor; and method steps may be performed by a programmable processor executing a program of instructions to perform functions of the described implementations by operating on input data and generating output.

The described features may be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that may be used, directly or indirectly, in a computer to perform a certain activity or bring about a certain result. A computer program may be written in any form of programming language (e.g., Objective-C, Java), including compiled or interpreted languages, and it may be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors or cores, of any kind of computer. Generally, a processor will receive instructions and data from a read-only memory or a random access memory or both. The essential elements of a computer are a processor for executing instructions and one or more memories for storing instructions and data. Generally, a computer may communicate with mass storage devices for storing data files. These mass storage devices may include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory may be supplemented by, or incorporated in, ASICs (application-specific integrated circuits).

To provide for interaction with a user the features may be implemented on a computer having a display device such as a CRT (cathode ray tube) or LCD (liquid crystal display) monitor for displaying information to the author and a keyboard and a pointing device such as a mouse or a trackball by which the author may provide input to the computer.

The features may be implemented in a computer system that includes a back-end component, such as a data server or that includes a middleware component, such as an application server or an Internet server, or that includes a front-end component, such as a client computer having a graphical user interface or an Internet browser, or any combination of them. The components of the system may be connected by any form or medium of digital data communication such as a communication network. Examples of communication networks include a LAN, a WAN and the computers and networks forming the Internet.

The computer system may include clients and servers. A client and server are generally remote from each other and typically interact through a network. The relationship of client and server arises by virtue of computer programs running on the respective computers and having a client-server relationship to each other.

One or more features or steps of the disclosed embodiments may be implemented using an Application Programming Interface (API). An API may define on or more parameters that are passed between a calling application and other software code (e.g., an operating system, library routine, function) that provides a service, that provides data, or that performs an operation or a computation.

The API may be implemented as one or more calls in program code that send or receive one or more parameters through a parameter list or other structure based on a call convention defined in an API specification document. A parameter may be a constant, a key, a data structure, an object, an object class, a variable, a data type, a pointer, an array, a list, or another call. API calls and parameters may be implemented in any programming language. The programming language may define the vocabulary and calling convention that a programmer will employ to access functions supporting the API.

In some implementations, an API call may report to an application the capabilities of a device running the application, such as input capability, output capability, processing capability, power capability, communications capability, etc.

As described above, some aspects of the subject matter of this specification include gathering and use of data available from various sources to improve services a mobile device can provide to a user. The present disclosure contemplates that in some instances, this gathered data may identify a particular location or an address based on device usage. Such personal information data can include location-based data, addresses, subscriber account identifiers, or other identifying information.

The present disclosure further contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. For example, personal information from users should be collected for legitimate and reasonable uses of the entity and not shared or sold outside of those legitimate uses. Further, such collection should occur only after receiving the informed consent of the users. Additionally, such entities would take any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices.

In the case of advertisement delivery services, the present disclosure also contemplates embodiments in which users selectively block the use of, or access to, personal information data. That is, the present disclosure contemplates that hardware and/or software elements can be provided to prevent or block access to such personal information data. For example, in the case of advertisement delivery services, the present technology can be configured to allow users to select to “opt in” or “opt out” of participation in the collection of personal information data during registration for services.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, content can be selected and delivered to users by inferring preferences based on non-personal information data or a bare minimum amount of personal information, such as the content being requested by the device associated with a user, other non-personal information available to the content delivery services, or publically available information.

A number of implementations have been described. Nevertheless, it will be understood that various modifications may be made. Elements of one or more implementations may be combined, deleted, modified, or supplemented to form further implementations. As yet another example, the logic flows depicted in the figures do not require the particular order shown, or sequential order, to achieve desirable results. In addition, other steps may be provided, or steps may be eliminated, from the described flows, and other components may be added to, or removed from, the described systems. Accordingly, other implementations are within the scope of the following claims.

1. A method comprising:
obtaining, at a computing device, sensor data generated by one or more accelerometers and one or more gyroscopes over a time period,
wherein the sensor data comprises:
an acceleration signal indicative of an acceleration measured by the one or more accelerometers over a time period, and
an orientation signal indicative of an orientation measured by the one or more gyroscopes over the time period, and
wherein the one or more accelerometers and the one or more gyroscopes are physically coupled to a user walking along a surface;
identifying, by the computing device, one or more portions of the sensor data based on one or more criteria; and
determining, by the computing device, characteristics regarding a gait of the user based on the one or more portions of the sensor data, wherein the characteristics comprise a walking speed of the user and an asymmetry of the gait of the user.

2. The method of claim 1, wherein the characteristics comprise a step length of the user.

3. The method of claim 1, wherein the characteristics comprise a percentage of time that both feet of the user are contacting the ground during a cycle of the gait of the user.

4. The method of claim 1, further comprising determining, based on the sensor data, the acceleration with respect to an inertial frame of reference.

5. The method of claim 1, wherein the characteristics regarding a gait of the user are estimated based on a pendulum model having the acceleration signal as an input.

6. The method of claim 1, the one or more portions of the sensor data are identified based on an estimated grade of the surface.

7. The method of claim 6, wherein the grade of the surface is estimated based on a barometer measurement obtained from a barometric sensor.

8. The method of claim 1, wherein the one or more portions of the sensor data are identified based on a comparison between the acceleration signal and a simulated acceleration signal.

9. The method of claim 8, wherein the simulated acceleration signal is determined based on a pendulum model.

10. The method of claim 1, wherein the one or more portions of the sensor data are identified based on an estimated activity type of the user during the time period.

11. The method of claim 1, wherein the one or more portions of the sensor data are identified based on a determination whether the user is performing a workout session.

12. The method of claim 1, wherein determining the asymmetry of the gait of the user comprises:
determining a plurality of steps taken by the user,
grouping pairs of steps into respective strides, and
determining the asymmetry of the gait of the user for each stride.

13. The method of claim 12, wherein determining the asymmetry of the gait of the user for each stride comprises determining a respective asymmetry score based on a logistic regression.

14. The method of claim 1, wherein the computing device comprises the one or more accelerometers and the one or more gyroscopes.

15. The method of claim 14, wherein the computing device is positioned asymmetrically about a center plane of the user.

16. A system comprising:
one or more processors;
memory storing instructions that when executed by the one or more processors, cause the one or more processors to perform operations comprising:
obtaining, at a computing device, sensor data generated by one or more accelerometers and one or more gyroscopes over a time period,
wherein the sensor data comprises:
an acceleration signal indicative of an acceleration measured by the one or more accelerometers over a time period, and
an orientation signal indicative of an orientation measured by the one or more gyroscopes over the time period, and
wherein the one or more accelerometers and the one or more gyroscopes are physically coupled to a user walking along a surface;
identifying, by the computing device, one or more portions of the sensor data based on one or more criteria; and
determining, by the computing device, characteristics regarding a gait of the user based on the one or more portions of the sensor data, wherein the characteristics comprise a walking speed of the user and an asymmetry of the gait of the user.

17.-30. (canceled)

31. One or more non-transitory, computer-readable storage media having instructions stored thereon, that when executed by one or more processors, cause the one or more processors to perform operations comprising:
obtaining, at a computing device, sensor data generated by one or more accelerometers and one or more gyroscopes over a time period,
wherein the sensor data comprises:
an acceleration signal indicative of an acceleration measured by the one or more accelerometers over a time period, and
an orientation signal indicative of an orientation measured by the one or more gyroscopes over the time period, and
wherein the one or more accelerometers and the one or more gyroscopes are physically coupled to a user walking along a surface;
identifying, by the computing device, one or more portions of the sensor data based on one or more criteria; and
determining, by the computing device, characteristics regarding a gait of the user based on the one or more portions of the sensor data, wherein the characteristics comprise a walking speed of the user and an asymmetry of the gait of the user.

32.-45. (canceled)
